(19) United States
(12) Patent Application Publication
(10) Pub. No.: US 2010/0228233 A1
(43) Pub. Date: Sep. 9, 2010

(54) MEDICAL DEVICE FOR INFUSION HAVING COILED TUBING OF ADJUSTABLE LENGTH

(76) Inventor: DARREN KAHN, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/781,602

(22) Filed: May 17, 2010

Related U.S. Application Data
(63) Continuation-in-part of application No. 29/254,422, filed on Feb. 23, 2006, now abandoned.

(51) Int. Cl. A61M 39/10

(52) U.S. Cl. 604/537; 604/533

(57) ABSTRACT

The embodiments of the subject invention in general describe an infusion device with adjustable length tubing. More particularly, the subject invention pertains to one or more embodiment(s) of an I.V. set, or similar device, having a coiled tubing. Specifically disclosed is an I.V. set with coiled tubing wherein the coils are attached but separable to add length and flexibility to the tubing.

### CROSS-REFERENCE TO RELATED APPLICATION

The present application is a continuation-in-part of and claims priority to U.S. Design patent application Ser. No. 29/254,422, filed Feb. 23, 2006, which is hereby incorporated by reference herein.

### BACKGROUND OF INVENTION

Disposable intravenous medical devices, often referred to as I.V. sets, are commonly used to administer or extract liquids from a patient. Such treatments are usually conducted through a vein punctured by a needle cannula element attached to one end of a long sterile flexible tube. The other end of the tube is often connected to a drip chamber attached to a pre-filled, sterile container, such as a glass or plastic bottle, but more often a plastic bag, of fluids with an attachment that allows the fluid being administered to a patient to flow one drop at a time.

The administering of blood or other vital fluids to a patient via an I.V. set is often conducted outside of a hospital or clinical setting. Emergency medical technicians, military medical technicians, and other first responders often work in confined spaces, such as ambulances, helicopters, or even in confined field situations during rescue operations. The elongated tubing often used with I.V. sets can be problematic to deal under such situations. It is vitally important that the tubing not get constricted or crimped. But, in preventing that, it often gets draped over the patient, other devices, or across the floor in these confined spaces, getting in the way of other procedures. In extreme situations, more than one I.V. set may be administered to a patient or multiple patients in the same space. Using shorter tubing can be helpful in confined spaces, but can be difficult to contend with after the patient reaches a hospital or clinic, where the staff often needs longer tubes to reach equipment or other devices. Once an I.V. is in place within a patient, it is not advisable to disturb the placement or interrupt the fluid flow to re-administer an I.V. with longer tubing.

Accordingly, there exists a need for I.V. sets or similar medical devices to have an adjustable tubing length. Ideally, such adjustable tubing will be easy to lengthen without crimping or constriction of the tubing or damage to the tubing that will affect the fluid flow.

### BRIEF SUMMARY

Embodiments of the subject invention are directed towards methods and devices for providing a disposable intravenous (I.V.) set that is safer and more convenient to use than standard I.V. sets currently in use. The embodiments of the subject invention successfully address the above described disadvantages associated with the previously known I.V. sets, and provide certain attributes and advantages, which have not been realized by the other known devices. The I.V. set embodiments disclosed herein provide novel, inexpensive, safe and convenient administration of fluids to a patient in both field and clinical settings.

In accordance with embodiments of the subject invention, the problem of non-adjustable length I.V. tubing is solved by incorporating coiled tubing with an I.V. set. More particularly, the disclosed embodiments relate to an I.V. set having coiled tubing with attached, but separable, coils that can be lengthened incrementally, as necessary. Unlike loose or unattached coils that engender an accordion-like effect, wherein the coils freely expand and contract, the coiled tubing disclosed herein is a rigid structure with coils that are immovable until they are unattached to provide a flexible tubing. To lengthen the tubing the coil structure can be broken apart at any point and the coils forcibly unwound or stripped off of the structure to add length to the flexible tubing. Alternatively, the tubing can be unwound or stripped off from either end of the coil structure to add length to the tubing.

Alternative embodiments described herein include multiple I.V. tubes coiled together, such that unwinding or stripping off the coils lengthens more than one I.V. tube. Particular embodiments include the same or different fluid intake or fluid administration attachments allowing the I.V. tubes to be used for similar or different purposes. In a more particular embodiment, the multiple I.V. tubes can be completely unwound and separated without disturbing fluid transmission to a patient. Further embodiments include various indicators, such as color and/or patterns on or within the tubing, to more easily distinguish between them.

### BRIEF DESCRIPTION OF DRAWINGS

In order that a more precise understanding of the above recited invention can be obtained, a more particular description of the invention briefly described above will be rendered by reference to specific embodiments thereof that are illustrated in the appended drawings. It should also be understood that the drawings presented herein may not be drawn to scale and that any reference to dimensions in the drawings or the following description are specific to the embodiments disclosed. Any variations of these dimensions that will allow the subject invention to function for its intended purpose are considered to be within the scope of the subject invention. Thus, understanding that these drawings depict only typical embodiments of the invention and are not therefore to be considered as limiting in scope, the invention will be described and explained with additional specificity and detail through the use of the accompanying drawings in which:

FIG. 1A is a front elevational view of a first embodiment of the design of the subject invention.

FIG. 1B is a left side elevational view of the first embodiment shown in FIG. 1.

FIG. 2 is a front elevational view of a second embodiment of the design of the subject invention.

FIG. 3 is a front elevational view of a third embodiment of the design of the subject invention.

FIG. 4 is a front elevational view of an embodiment of a multiple coil attached tube structure of the subject invention.

### DETAILED DISCLOSURE

The embodiments of the subject invention in general describe an infusion device with adjustable length tubing. More particularly, the subject invention pertains to one or more embodiment(s) of an I.V. set, or similar device, having a coiled tubing. Specifically disclosed is an I.V. set with coiled tubing wherein the coils are attached but separable to add length and flexibility to the tubing.

The following description will disclose that the subject invention is particularly useful in the field of medical first responders, in particular medical first responders working in confined, often moving or transporting environments, such, for example an ambulance or helicopter. However, a person with skill in the art will be able to recognize numerous other uses that would be applicable to the devices and methods of the subject invention. While the subject application describes a use for fluid transmission to a patient, other modifications apparent to a person with skill in the art and having benefit of the subject disclosure are contemplated to be within the scope of the present invention.

In the description that follows, a number of terms used related to medical devices, I.V. sets in particular, are utilized. In order to provide a clear and consistent understanding of the specification and claims, including the scope to be given such terms, the following definitions are provided.

The term “patient” as used herein, describes an animal, including mammals to which the systems and methods of the present invention are applied. Mammalian species that can benefit from the disclosed systems and methods include, but are not limited to, apes, chimpanzees, orangutans, humans, monkeys; domesticated animals (e.g., pets) such as dogs, cats, guinea pigs, hamsters; veterinary uses for large animals such as cattle, horses, goats, sheep; and any wild animal for veterinary or tracking purposes.

The term “medical technician” or “technician” as used in the subject application is merely for literary convenience. The term should not be construed as limiting in any way. The devices, apparatuses, methods, techniques and/or procedures disclosed herein could be utilized by any person desiring or needing to do so and having the necessary skill and understanding of the invention, including, but not limited to, doctors, nurses, medical first responders, and similarly trained individuals.

Also, as used herein, and unless otherwise specifically stated, the terms “operable communication” and “operably connected” and the like mean that the particular elements are connected in such a way that they cooperate to achieve their intended function or functions. The “connection” may be direct, or indirect, physical or remote.

In addition, references to “first”, “second”, and the like (e.g., first and second ends), as used herein, and unless otherwise specifically stated, are intended to identify a particular feature of which there can be at least two. Such reference to “first” does not imply that there must be two or more. However, these references are not intended to confer any order in time, structural orientation, or sidedness (e.g., left or right) with respect to a particular feature.

Finally, reference is made throughout the application to the “proximal end” and “distal end.” As used herein, the proximal end is that end nearest the patient or from which a fluid being administered exits the tubing. Conversely, the distal end of the device is that end furthest from the patient or into which a fluid to be administered enters the tubing.

The present invention is more particularly described in the following examples that are intended to be illustrative only since numerous modifications and variations therein will be apparent to those skilled in the art. As used in the specification and in the claims, the singular for “a,” “an” and “the” include plural referents unless the context clearly dictates otherwise.

With reference to the attached figures, which show certain embodiments of the subject invention, it can be seen that the intravenous set 10 disclosed herein can include a tubing 19 wherein all or some portion thereof is arranged as attached coil tubing 20 having affixed to one or both ends various fluid intake 30 and fluid control 40 mechanisms, as well as fluid transfer mechanisms 50. In addition, various types of accessory parts can be placed along and/or in-line with the tubing for attachment of other devices or injection of other materials into the tubing.

With regard to the attached coil tubing 20, the figures illustrate an embodiment wherein a flexible I.V. tubing is configured into attached helical coils forming a generally linear structure. In this embodiment, the individual helical coils 22 of the tubing are attached substantially parallel to each other to create a substantially rigid structure 21. The attachment of the coils can prohibit flexibility between them, that is, they do not expand and contract or otherwise exhibit an “accordion-like” behavior. This can provide better control over and placement of the tubing and less opportunity for crimping or constriction. However, when the coils are unattached or unwound, as described below, the tubing 19 becomes generally flexible.

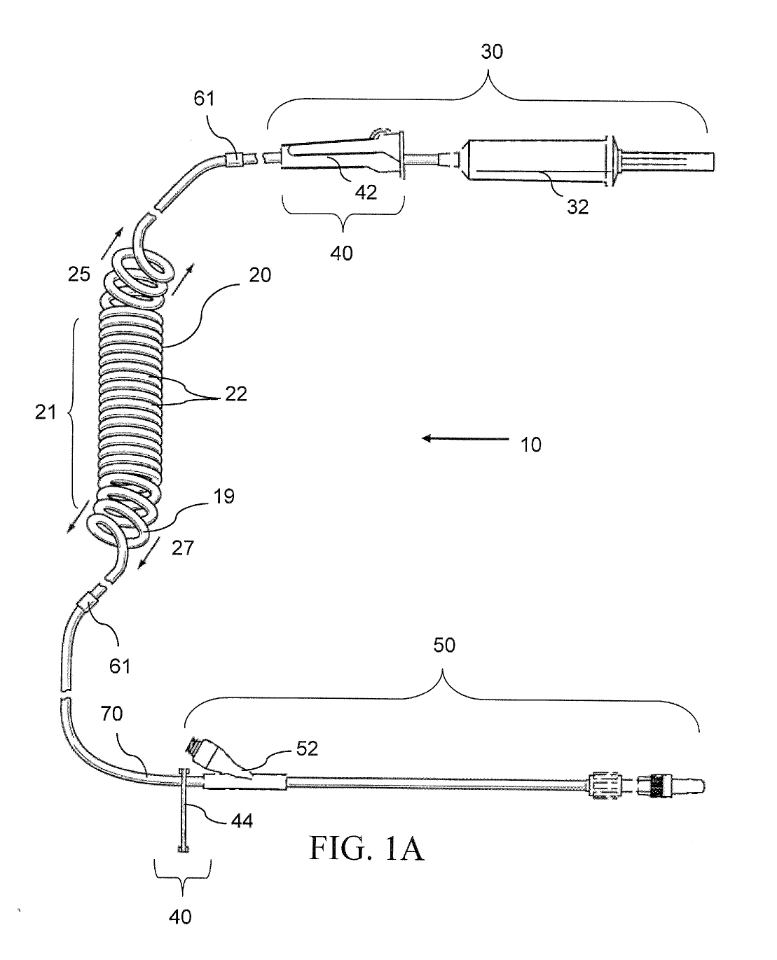

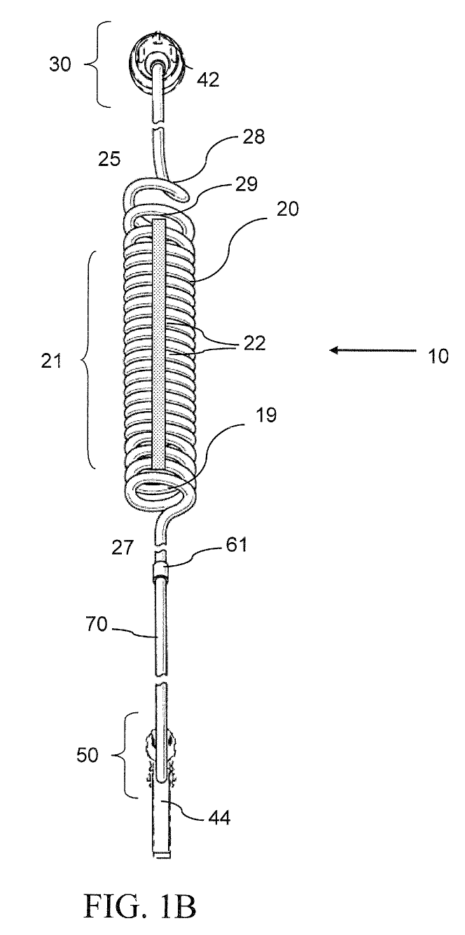

The individual coils of the attached coil tubing 20 can have any of a variety of circumferential shapes including, but not limited to, circular, oval, triangular, square, rectangular, and any other polygonal shape or combination thereof. In a specific embodiment, shown in FIGS. 1-3, the individual coils 22 have a circumferential shape, such that, when attached the overall shape of the attached coil tubing 20 resembles a circular tube.

There are any of a variety of methods and techniques that can be employed to attach the coils of the embodiments disclosed herein. Whichever method is employed, it can be beneficial for it to ensure that the coils maintain all or at least part of their rigid structure 21 during packaging, transport, and general usage. Such method should also permit the coils to be released individually by breaking apart or bending the rigid structure between coils or by stripping, unwinding, pulling, or otherwise forcibly disconnecting the coils from one another to provide a sufficiently flexible tubing. In a particular embodiment, the coils can be released by pulling on a first end 25 and/or a second end 27, as indicated by the arrows in FIGS. 1-3, causing the tubing to be pulled off or unwound incrementally. In a preferred embodiment, the separation of the coils will not damage the integrity of the tubing or its ability to transport fluids. In other words, pulling the tubing from either end or breaking or bending the rigid structure 21 will release coils, but will not cause ripping, tearing or shredding of the tubing that will cause undesirable openings or holes.

The attachment of the coils in the attached coil tubing can be accomplished by several methods and techniques. In one embodiment, the coils are attached by the use of one or more adhesives. The adhesive can be applied to most or all of the exterior surface of the coil structure prior to the individual coils being attached. In an alternative embodiment, the adhesive can be applied only to strategic points along the exterior of the coils that will permit them to be adhered together.

Alternative embodiments can utilize one or more adhesive strips 12 or a similar product placed on the exterior surface of the coils. In one embodiment, shown, for example, in FIG. 1B, the adhesive strip 12 can be placed against the outer edges 28 of exterior surface of the coils. Alternatively, the adhesive strip can be placed against the inner edge 29 of the exterior surface of the coils. In a particular embodiment, at least four adhesive strips are placed against the inner edge 29 of the exterior surface of the coils.

Still further alternative embodiments can employ one or more manufacturing methods that allow the coils to be attached without the use of adhesives or other secondary materials. In one embodiment, one or more of the materials utilized in the flexible tubing can perform as an adhesive or have adhesive-like properties for a time sufficient during manufacture for the coils to be adhered to one another. A person with skill in the art would be able to devise any of a multitude of methods, materials and/or devices for adhering the coils of an attached coil tubing 20, in accordance with the embodiments of the subject invention. It should be understood that any and all such variations are considered to be within the scope of the subject invention.

After one or more coils have been detached from the rigid structure 21 the tubing can be extended or straightened to the necessary length. Sometimes it can be preferable for the tubing to be only of a particular length so that there is little or no excess movement. Thus, in one embodiment, as the tubing is released from the rigid structure it forms a flexible, generally straight length of tubing with little or no shape memory of the previous coiled configuration. However, in other situations, it can be beneficial for the flexible tubing to maintain some or all of the coiled configuration even when unattached. This can allow the tubing to extend and retract adding or subtracting length as necessary without releasing more than necessary from the rigid structure 21. In one embodiment, the tubing has some shape memory so that individual coils 20 partially retain the coiled configuration, even when separated from the rigid structure 21.

Advantageously, embodiments of the attached coil tubing of the subject invention engender I.V. sets, or other devices with which it may be utilized, with a compact, easily transportable, and storable configuration. The overall length of the tubing when coiled and when fully uncoiled can vary depending upon the specific needs of a technician. In one embodiment, the overall length of the attached coil tubing when attached as a rigid structure 21 is between approximately 2.0 inches and approximately 6.0 inches. In a more particular embodiment, the overall length of the attached coil tubing when attached as a rigid structure 21 is between approximately 3.0 inches and approximately 5.0 inches. In a specific embodiment, the overall length of the attached coil tubing when attached as a rigid structure 21 is approximately 4.5 inches.

The extended length of the tubing after all of the attached coils are unwound can depend upon not only the length of the attached coil tubing, but the diameter of the helical coils. As mentioned above, the embodiments of the subject invention are compact, easy to transport, and, importantly, easy to store in small compartments and spaces typically utilized by field medical technicians. In one embodiment, the diameter of the helical coils is between 0.5 inches and 2.0 inches. In a more particular embodiment, the diameter of the helical coils is between 0.75 inch and 1.5 inch. In a specific embodiment, the diameter of the helical coils is approximately 1.0 inch.

The total length of the tubing incorporated into a rigid structure 21 can depend upon the above-mentioned factors. It can be important for the tubing to be sufficiently adjustable in length to accommodate for use in not only an emergency setting, often under limited space conditions, but also in a hospital or clinical setting where longer tubing may be necessary. In one embodiment, the tubing incorporated into a rigid structure can be expanded to a length of between approximately 2.0 feet and approximately 8.0 feet. In a more particular embodiment, the tubing incorporated into a rigid structure can be expanded to a length of between approximately 3.0 feet and approximately 7.0 feet. In still more particular embodiment, the tubing incorporated into a rigid structure can be expanded to a length of between approximately 4.0 feet and approximately 6.0 feet. In a specific embodiment, the tubing incorporated into a rigid structure can be expanded to a length of approximately 6.5 feet.

There can arise situations where more than one I.V. tubing is required, either for a single patient or for multiple patients. In a crowded or confined space, the use of multiple tubes can be confusing and/or difficult to work around, particularly if they are overly long. The attached coil tubing embodiments disclosed herein are advantageous is controlling such confusion and provide a safer work environment both for medical technicians as well as patients. However, to further control situations in which multiple tubes are required, it can be beneficial if they can be combined into a single unit.

Embodiments of the subject invention can include two or more flexible tubes joined, but separable along their length to form a single elongated structure of individual tubings. If required, the individual tubings can be fully or partially separated, such as, for example, as described above, to provide partially or fully individual tubings.

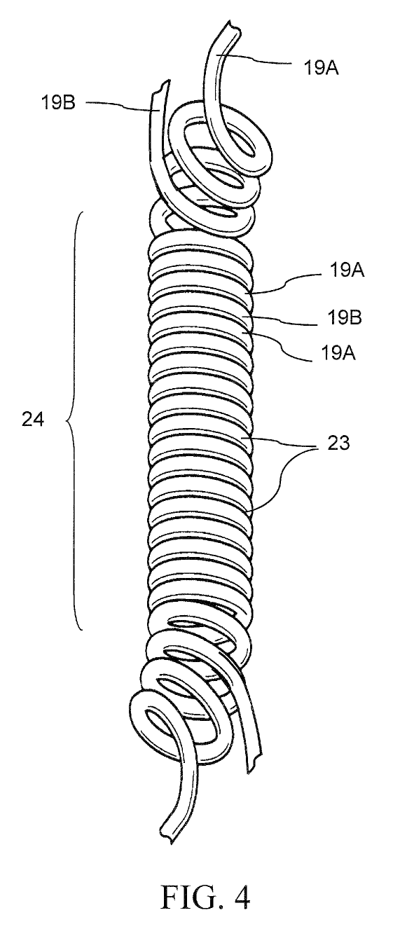

In a further embodiment, the two or more flexible tubings can be coiled together to form a single rigid structure 23, an example of which is shown in FIG. 4. In this embodiment, the tubes are coiled together such that the helical coils of each tube alternate with and are parallel to the helical coils of one or more other tubes in the same rigid structure 23. As with a single attached coil tubing, individual coils 22 can be separated to provide a desired length. In a specific embodiment, shown, for example, in FIG. 4, two separate tubings 19A and 19B are arranged into an attached coil tubing 23. In a further embodiment, the coils alternate in the attached coil tubing.

In one embodiment, the length of each tube utilized in a multiple coil rigid structure 24 is approximately the same. In an alternative embodiment, the length of each tube utilized in a multiple coil rigid structure is substantially different, such that one tube may be shorter than one or more other tubes configured into the multiple coil rigid structure 24. In a particular embodiment, the tubes are coiled such that when the rigid structure 23 is fully unwound, the individual tubes 19A and 19B are not twisted around one another and can be easily moved apart and/or separated without disconnecting from a patient or an I.V. fluid container.

To facilitate identification of the individual tubes of a multiple coil tube I.V. set, each tube can have distinctive, individual markings or coloration. This can help to ensure that each tube is correctly used and/or attached to the proper patient and/or I.V. fluid container. It can also assist in the later separation of the tubes if a multiple coil I.V. set is used for more than one patient or if one of the tubes needs to be removed or adjusted on the same patient.

There are a variety of accessory parts 60 that can be permanently, removably, or moveably attached to I.V. tubing that can be necessary or useful for fluid intake, fluid transfer, or fluid control into and through the tubing 19. By way of non-limiting example, fluid intake mechanisms 30 can include drip chambers, spikes, Y-connectors that can allow piggy-backing of IV fluid sets or injection of other fluids into the IV tubing. Fluid transfer mechanisms 50 can include such non-limiting examples as hollow needles or syringes, cannulas, catheters, hubs and/or injection ports used to administer additional medicines to a patient through the tubing. In some embodiments, fluid intake mechanisms and fluid transfer mechanisms can be the same or similar type components. For example, various types of Y-connectors can be used as part of the fluid intake or fluid transfer process. Further, fluid control mechanisms 40 can include, by way of non-limited examples, roller clamp, luer slip, slide clamp, on/off clamp, and line clamps. Also useable with the embodiments herein are line connectors 61 for connecting different sections of tubing together, spike hanger for securing tubing to an IV fluid container and other devices known to those with skill in the art. Other accessory parts that can be utilized with the embodiments herein include, but are not limited to, Pressure Activated Valves, Back Check Valves; Luer Activated Valves; Pre-Slit Blunt Cannula Valves;Anesthesia Manifolds; Flow Control Devices; Gravity Flow Controller; Disposable/Portable IV Infusion Pump; Roller Clamps/Pinch Clamps/Slide Clamps; Pre; Attached IV Extension Sets; Y Connector; Medication Injection Site; Line Identification Markers; Drip Chamber/Spikes; Filters/Filter Assemblies; and/or Burette Mixer/Blood Chamber.

The embodiments of the attached coil IV set of the subject invention can be manufactured as a single tube, wherein the coiled portion is contiguous with the tubing connected to the accessory components or parts. In other embodiments, the coiled portion of the IV set can be manufactured as a separate component that can be attached at one or both ends to the IV tubing utilized with the accessory parts 60. For example, in FIG. 1A, it can be seen that the attached coil IV tubing 20 is connected to a length of non-coiled IV tubing 70 which is used to convey fluids from the fluid intake mechanisms 30. At the proximal end, the attached coiled tubing is connected to a second length of non-coiled IV tubing 70 that leads to the accessories for fluid transfer 50 to a patient. The connection point between the coiled tubing and the non-coiled tubing can be accomplished by any of a variety of techniques. In the embodiment shown for example in FIG. 1A, a line connector 61 is utilized to attach an end of non-coiled tubing to an end of the coiled tubing. In a further embodiment, one or more suitable adhesives, such as, for example, a biocompatible adhesive, can be used in conjunction with a line connector 61 to securely join and affix the two ends.

Following are examples that illustrate procedures for practicing the subject invention. These examples are provided for the purpose of illustration only and should not be construed as limiting. Thus, any and all variations that become evident as a result of the teachings herein or from the following examples are contemplated to be within the scope of the present invention.

### Example 1

FIGS. 1A and 1B illustrate a specific embodiment of an attached coil IV set 10 of the subject invention. In this embodiment, connection of the set to an IV fluid container is facilitated by the use of a drip chamber 32 affixed to the distal end with an attached spike for penetrating the fluid container. To control fluid flow, a roller clamp 42 can be positioned proximal to the drip chamber. In this embodiment, the non-coiled IV tubing 70 is connected to the distal end of the attached coil tubing 20 by use of a connector 61.

At the proximal end of the attached coil IV tubing a second line connector 61 used to attach a length of non-coiled tubing 70 that leads to the fluid transfer mechanisms 50. In this embodiment, a slide clamp is used proximal to the connector to control the flow of fluid to the patient. A needleless-valve Y-connector 52 can be attached proximal to the slide clamp 44 to facilitate the introduction of additional components into the IV tubing just prior to fluid transfer to a patient. At the most proximal end is a needle and/or catheter with a luer connection for injection into a patient.

### Example 2

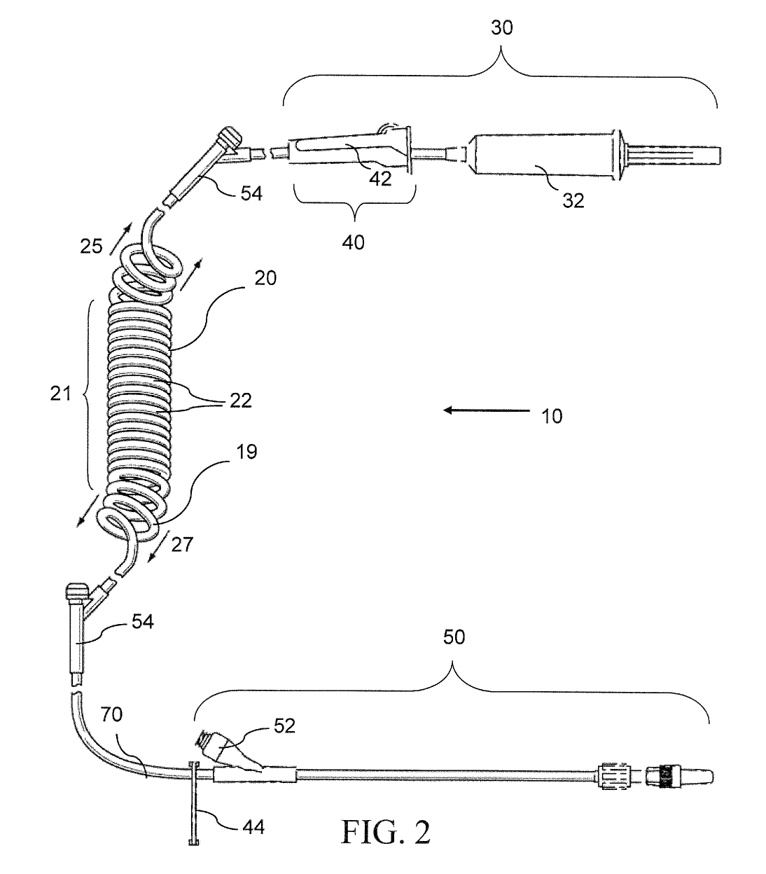

FIG. 2 illustrates another specific embodiment of an attached coil IV set 10 of the subject invention. In this embodiment, connection to an IV fluid container is facilitated by the use of a drip chamber 32 affixed to the distal end with an attached spike for penetrating the fluid container. To control fluid flow, a roller clamp 42 can be positioned proximal to the drip chamber. In this embodiment, the non-coiled IV tubing 70 is connected to the distal end of the attached coil tubing 20 by use of an injection ported Y-connector 54.

At the proximal end of the attached coil IV tubing a second injection ported Y-connector 54 is used to attach a length of non-coiled tubing 70 that leads to the fluid transfer mechanisms 50. In this embodiment, a slide clamp 44 is used proximal to the Y-connector to control the flow of fluid to the patient. A needleless-valve Y-connector 52 can be attached proximal to the slide clamp to facilitate the introduction of additional components into the IV tubing without the use of a syringe, just prior to fluid transfer to a patient. At the most proximal end is a needle and/or catheter with a luer connection for injection into a patient.

### Example 3

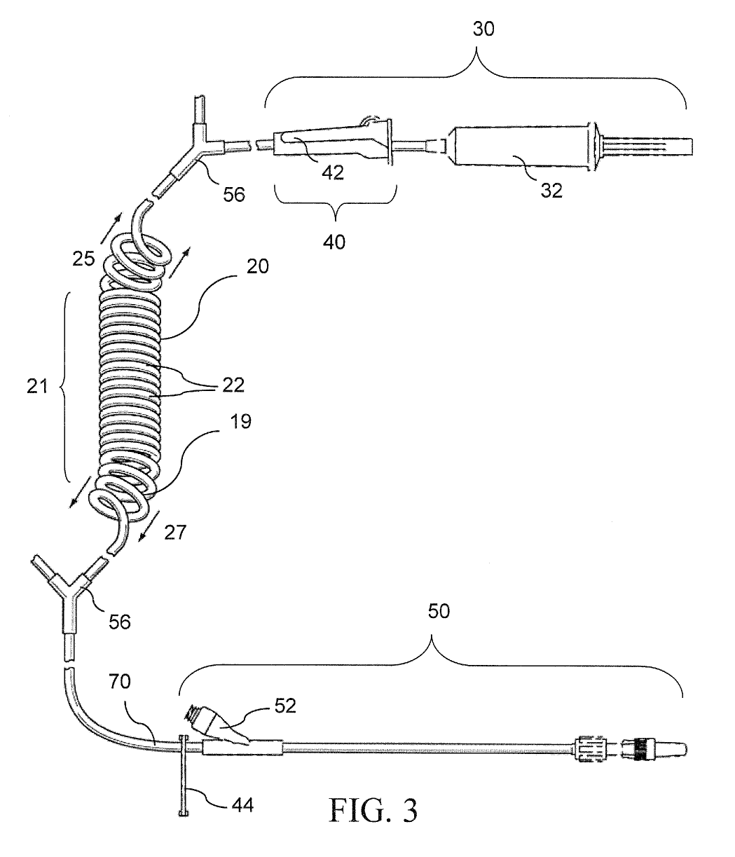

FIG. 3 illustrates a further specific embodiment of an attached coil IV set 10 of the subject invention In this embodiment, connection to an IV fluid container is facilitated by the use of a drip chamber 32 affixed to the distal end with an attached spike for penetrating the fluid container. To control fluid flow, a roller clamp 42 can be positioned proximal to the drip chamber. In this embodiment, the non-coiled IV tubing 70 is connected to the distal end of the attached coil tubing 20 by use of a piggy-backY-connector 56.

At the proximal end of the attached coil IV tubing a second piggy-back Y-connector 56 is used to attach a length of non-coiled tubing 70 that leads to the fluid transfer mechanisms 50. In this embodiment, a slide clamp 44 is used proximal to the Y-connector to control the flow of fluid to the patient. Further, a needleless-valve Y-connector can be attached proximal to the slide clamp to facilitate the introduction of additional components into the IV tubing just prior to fluid transfer to a patient. At the most proximal end is a needle and/or catheter with a luer connection for injection into a patient.

All patents, patent applications, provisional applications, and other publications referred to or cited herein are incorporated by reference in their entirety, including all figures and tables, to the extent they are not inconsistent with the explicit teachings of this specification. Additionally, the entire contents of the references cited within the references cited herein are also entirely incorporated by reference.

It should be understood that the examples and embodiments described herein are for illustrative purposes only and that various modifications or changes in light thereof will be suggested to persons skilled in the art and are to be included within the spirit and purview of this application.

It should be understood that any reference in this specification to “one embodiment,” “an embodiment,” “example embodiment,” “further embodiment,” “alternative embodiment,” etc., is for literary convenience. The implication is that any particular feature, structure, or characteristic described in connection with such an embodiment is included in at least one embodiment of the invention. The appearance of such phrases in various places in the specification does not necessarily refer to the same embodiment. Further, when a particular feature, structure, or characteristic is described in connection with any embodiment, it is submitted that it is within the purview of one skilled in the art to affect such feature, structure, or characteristic in connection with other ones of the embodiments.

The invention has been described herein in considerable detail, in order to comply with the Patent Statutes and to provide those skilled in the art with information needed to apply the novel principles, and to construct and use such specialized components as are required. However, it is to be understood that the invention can be carried out by specifically different equipment and devices, and that various modifications, both as to equipment details and operating procedures can be effected without departing from the scope of the invention itself. Further, it should be understood that, although the present invention has been described with reference to specific details of certain embodiments thereof and by examples disclosed herein, it is not intended that such details should be regarded as limitations upon the scope of the invention except as and to the extent that they are included in the accompanying claims.

I claim:

1. An intravenous medical device comprising:
an elongated tubing having a distal end and a proximal end, where some portion therebetween is formed as a rigid coil structure of separably attached coils;
one or more fluid intake mechanisms affixed to the distal end of the tubing;
one or more fluid transfer mechanisms affixed to the proximal end of the tubing.

2. An intravenous medical device, according to claim 1, further comprising one or more fluid control mechanisms.

3. An intravenous medical device comprising:
two or more elongated tubings having a distal end and a proximal end, where for some portion therebetween the two or more elongated tubings are joined to form a single rigid coil structure of separably attached coils;
one or more fluid intake mechanisms affixed to the distal end of one or more of the tubings;
one or more fluid transfer mechanisms affixed to the proximal end of one or more of the tubings.

4. An intravenous medical device, according to claim 1, further comprising one or more fluid control mechanisms.
